SEQUENCE LISTING (16 sequences)

SEQ ID NO: 1 - DNA, 25 bp, Artificial sequence
  Synthesized
caccgttcaatctctgtgtttgaag

SEQ ID NO: 2 - DNA, 25 bp, Artificial sequence
  Synthesized
aaaccttcaaacacagagattgaac

SEQ ID NO: 3 - DNA, 25 bp, Artificial sequence
  Synthesized
caccgacgatctgctcgagtcaccc

SEQ ID NO: 4 - DNA, 25 bp, Artificial sequence
  Synthesized
aaacgggtgactcgagcagatcgtc

SEQ ID NO: 5 - DNA, 25 bp, Artificial sequence
  Synthesized
caccgacggaggctaagcgtcgcaa

SEQ ID NO: 6 - DNA, 25 bp, Artificial sequence
  Synthesized
aaacttgcgacgcttagcctccgtc

SEQ ID NO: 7 - DNA, 19 bp, Artificial sequence
  Synthesized
atggagccttcggctgact

SEQ ID NO: 8 - DNA, 21 bp, Artificial sequence
  Synthesized
gtaactattcggtgcgttggg

SEQ ID NO: 9 - DNA, 22 bp, Artificial sequence
  Synthesized
cgatggaacttcgactttgtca

SEQ ID NO: 10 - DNA, 21 bp, Artificial sequence
  Synthesized
gcacaagggtacaagacagtg

SEQ ID NO: 11 - DNA, 22 bp, Artificial sequence
  Synthesized
gaaaaagacaactctcgtcgca

SEQ ID NO: 12 - DNA, 23 bp, Artificial sequence
  Synthesized
gtaagcactgatttccatgtcca

SEQ ID NO: 13 - DNA, 20 bp, Artificial sequence
  Synthesized
cccctcggtcctgacaaaag

SEQ ID NO: 14 - DNA, 20 bp, Artificial sequence
  Synthesized
cgctcttcgtcactggagaa

SEQ ID NO: 15 - DNA, 25 bp, Artificial sequence
  Synthesized
caccgagccgccggcaatgccgcga

SEQ ID NO: 16 - DNA, 25 bp, Artificial sequence
  Synthesized
aaactcgcggcattgccggcggctc

(19) United States
(12) Patent Application Publication
(10) Pub. No.: US 2023/0416712 A1
(43) Pub. Date: Dec. 28, 2023

(54) APPLICATION OF SUBSTANCE THAT REDUCES THE CONTENT OR ACTIVITY OF KAT7 IN PREVENTING SENESCENCE AND TREATING HEPATIC FIBROSIS

(71) Applicant: INSTITUTE OF ZOOLOGY, CHINESE ACADEMY OF SCIENCES, Beijing (CN)

(72) Inventors: Guanghui LIU, Beijing (CN); Jing QU, Beijing (CN); Weiqi ZHANG, Beijing (CN); Wei WANG, Beijing (CN); Shuhui SUN, Beijing (CN); Wei LI, Beijing (CN); Moshi SONG, Beijing (CN); Si WANG, Beijing (CN)

(21) Appl. No.: 18/247,058

(22) PCT Filed: Jun. 4, 2021
(86) PCT No.: PCT/CN2021/098217

§ 371 Date: Mar. 28, 2023

(30) Foreign Application Priority Data
Sep. 29, 2020 (CN) 202011048122.X

(51) Int. Cl. C12N 9/22; C12N 15/63; A61K 38/00; A61P 1/16

(52) U.S. Cl. CPC C12N 9/22; C12N 15/63; A61K 38/005; A61P 1/16

(57) ABSTRACT

The present invention discloses an application of a substance that reduces the content or activity of KAT7 in preventing senescence and treating hepatic fibrosis. The present invention finds out that knockdown KAT7 genes have a definite effect on delaying senescence and treating hepatic fibrosis; and WM-3835 as a KAT7 inhibitor has a definite effect on delaying the senescence of hepatocytes. The present invention provides a new idea for developing genes for delaying senescence and treating hepatic fibrosis, and expands the options of clinical gene therapy and drug therapy.

### RELATED APPLICATIONS

The present application is a U.S. National Phase of International Application Number PCT/CN2021/098217 filed Jun. 4, 2021, and claims priority to Chinese Application Number 202011048122.X filed Sep. 29, 2020.

### INCORPORATION BY REFERENCE

The sequence listing provided in the file entitled C6351-099_SQL_v1.txt, which is an ASCII text file that was created on Mar. 28, 2023, and which comprises 3,323 bytes, is hereby incorporated by reference in its entirety.

### TECHNICAL FIELD

The present invention relates to the field of biomedicine, in particular to an application of a substance that reduces the content or activity of KAT7 in preventing senescence and treating hepatic fibrosis.

### BACKGROUND

Senescence in nature is a process of gradual decline in functions of various organ systems in a body, which will finally result in death of an individual. The degenerative changes in an organ function are driven by both genetic and environmental factors. With the senescence of a body, cells as the smallest functional units that make up the tissues and the organs also become senescent. Cell senescence refers to a changing process in which the cell proliferation and differentiation abilities and physiological functions gradually decline with the passage of time while cells executing life activities. A certain number of senescent cells accumulate in senescent organs and tissues, and the accumulated senescent cells have lost their original normal physiological functions, thereby affecting the functions of the organs and tissues. Meanwhile, the senescent cells will secrete a large number of inflammatory factors called senescence-associated secretory phenotype (SASP), and the production of the SASP will deteriorate the tissue microenvironment. However, individual senescence or senescence-associated degenerative changes can be delayed by taking drugs or by means of a gene therapy to eliminate senescent cells or delay the senescence of cells.

Cell senescence as an inevitable process of life is affected by both genetic and environmental factors. Forward genetic screening is an ideal tool to determine the human genes that are involved in a specific biological process. The CRISPR/Cas9 gene editing technique developed in recent years can deliberately cut a target genome to inactivate the corresponding gene function. Therefore, it can be applied in screening senescence-associated genes.

Hepatic fibrosis is a senescence-associated disease, and hepatic fibrosis is more severe in elderly individuals than in younger individuals. The specific manifestations are the regeneration of hepatocytes after repeated destruction, and the diffuse excessive deposition and abnormal distribution of collagen, glycoproteins, proteoglycans and other extracellular matrix in the liver. It is the pathological repair response of liver to a chronic injury. Hepatic fibrosis is histologically reversible, but it can progress to cirrhosis without treatment. The only treatment for cirrhosis is liver transplantation, however, due to the shortage of donors, the vast majority of the patients are not well treated. Therefore, the period of hepatic fibrosis is a window period for treatment. It has been reported that CCL4 (carbon tetrachloride)-induced hepatic fibrosis can be alleviated by inducing the factors such as Foxa1, Foxa2, Foxa3, Gata4, Hnf1a and Hnf4a into the liver by means of gene therapy.

### SUMMARY

The present invention is intended to solve the technical problems of preventing senescence and treating hepatic fibrosis.

In order to solve the above technical problems, the present invention provides any of the following applications:
1. an application of a substance that reduces the content or activity of KAT7 in preparing a product for treating and/or preventing senescence;
2. an application of a substance that inhibits KAT7 gene expressions or knocks out KAT7 genes in preparing a product for treating and/or preventing senescence.

In the above applications, the substance can be a CRISPR/Cas9 gene knockout system targeting KAT7 genes.

In the above applications, the substance can be a recombinant vector containing the CRISPR/Cas9 gene knockout system.

The recombinant vector can be a sgRNA plasmid that can express a specific targeting KAT7 gene obtained by inserting a specific fragment of the KAT7 gene into lenti-CRISPRv2 using Esp3I.

In the above applications, the substance can be a KAT7 inhibitor.

Specifically, the KAT7 inhibitor can be WM-3835.

In the above applications, the senescence can be the senescence of an animal cell, a tissue, an organ or an individual.

The senescence of cells can be that of mesenchymal precursor cells or mesenchymal stem cells, or that of hepatocytes.

The senescence of mesenchymal precursor cells or mesenchymal stem cells can be pathological senescence or replicative senescence.

In an embodiment of the present invention, the pathological senescence of the mesenchymal precursor cells or mesenchymal stem cells refers to the senescence caused by the mutation of human progeria genes (WRN loss of function).

In an embodiment of the present invention, the senescence of hepatocytes refers to the senescence of primary cells.

The senescence of a tissue or an organ may be the natural senescence of a tissue or an organ.

The senescence of an individual can be natural aging.

The animal can be a mammal, such as a human or a mouse.

The present invention also provides an application of a substance that reduces the content or activity of KAT7 or a substance that inhibits KAT7 gene expressions or knocks out KAT7 genes in preparing a product for inhibiting hepatic fibrosis.

In an embodiment of the present invention, the hepatic fibrosis is CCL4-induced hepatic fibrosis.

The present invention also provides a product for treating and/or preventing senescence, and an active ingredient of the product can be a substance that reduces the content or activity of KAT7 or a substance that inhibits KAT7 gene expressions or knocks out KAT7 genes.

The present invention also provides a product for inhibiting hepatic fibrosis, and an active ingredient of the product can be a substance that reduces the content or activity of KAT7 or a substance that inhibits KAT7 gene expressions or knocks out KAT7 genes.

The present invention also provides a method for delaying and/or treating and/or preventing senescence, and the method includes the following steps: administrating a substance that reduces the content or activity of KAT7 or a substance that inhibits KAT7 gene expressions or knocks out KAT7 genes on an animal cell, a tissue, an organ or an individual, in order to delay and/or treat and/or prevent the senescence of the animal cell, the tissue, the organ or the individual.

In the above method, the animal cell, the tissue, the organ or the individual can be either a normal animal cell, a normal tissue, a normal organ or a normal individual, or a senescent animal cell, a senescent tissue, a senescent organ or a senescent individual.

The animal can be a mammal. The mammal can be a human or a mouse.

The cells can be mesenchymal precursor cells or mesenchymal stem cells or hepatocytes.

The senescence of mesenchymal precursor cells or mesenchymal stem cells can be pathological senescence or replicative senescence.

In an embodiment of the present invention, the pathological senescence of mesenchymal precursor cells or mesenchymal stem cells refers to the senescence caused by the mutation of human progeria genes (WRN loss of function).

In an embodiment of the present invention, the senescence of hepatocytes refers to the senescence of primary cells.

The senescence of a tissue or an organ may be the natural senescence of a tissue or an organ.

The senescence of an individual can be natural aging.

In the present invention, the product can be a drug. The mesenchymal cells can be mesenchymal precursor cells or mesenchymal stem cells.

### BRIEF DESCRIPTION OF THE DRAWINGS

FIGS. 1A-1E show that knockdown KAT7 genes can delay an accelerated senescent phenotype of mesenchymal precursor cells carrying human progeria gene mutation. sg-NTC represents the cells infected with sgNTC virus, and sg-KAT7 #1 and sg-KAT7 #2 represent two types of cells infected with sgKAT7-human lentivirus. The Bar value in b is 25 mm. *** represents a significant difference (p<0.001).

FIGS. 2A-2C show that knockdown KAT7 genes can delay the replicative senescence of mesenchymal precursor cells. sg-NTC represents the cells infected with sgNTC virus, and sg-KAT7 represents the cells infected with sg-KAT7 #1 lentivirus. The Bar values in a and b are 25 mm and 100 mm. *** represents a significant difference (p<0.001).

FIGS. 3A-3H show the therapeutic effect of knockdown KAT7 genes on delaying senescence in an aged mouse. Young represents a young mouse, Aged represents an aged mouse, sg-NTC represents an aged mouse infected with sgNTC virus, and sg-KAT7 represents an aged mouse infected with sgKAT7-mouse lentivirus. The Bar values in f are 300 mm and 200 mm. * represents a significant difference (p< and ** represents a significant difference (p<0.01).

FIGS. 4A-4F show that knockdown KAT7 genes can delay the senescence of human primary hepatocytes. sg-NTC represents the hepatocytes infected with sgNTC virus, and sg-KAT7 represents the hepatocytes infected with sgKAT7-human lentivirus. The Bar value in b is 100 mm. *** represents a significant difference (p< and ** represents a significant difference (p<0.01).

FIGS. 5A and 5B show the therapeutic effect of knockdown KAT7 genes on delaying CCL4-induced hepatic fibrosis. sg-NTC represents a mouse infected with sgNTC virus, and sg-KAT7 represents a mouse infected with sgKAT7-mouse lentivirus. The Bar values in a in a downward order are 300 mm, 50 mm, 100 mm and mm. * represents a significant difference (p<0.05), ** represents a significant difference (p<0.01), and *** represents a significant difference (p<0.001).

FIGS. 6A-6I show an effect of WM-3835 as a KAT7 inhibitor in delaying the senescence of mesenchymal precursor cells and hepatocytes. DMSO represents the mesenchymal precursor cells or hepatocytes treated by a control product, and WM-3835 represents the mesenchymal precursor cells or hepatocytes treated by the inhibitor. The Bar value in b is 25 mm, and the Bar value in c is 50 mm. ** represents a significant difference (p<0.01), and *** represents a significant difference (p<0.001).

### DETAILED DESCRIPTION OF THE EMBODIMENTS

The present invention will be described further below in combination with specific embodiments, and the embodiments are given only for illustrating the present invention, rather than limiting the scope of the present invention. The embodiments below can be used as a guidance for those of ordinary skill in the art to make further improvement but will not limit the scope of the present invention in any way.

Unless otherwise specified, all the experimental methods in the following embodiments are conventional methods and shall be performed in accordance with the techniques or conditions described in the reference in the art or the product manual. Unless otherwise specified, all the materials, reagents and instruments used in the following embodiments are commercially available. Three repeated experiments are established for the quantitative tests in the following embodiments. All the animal experiments are approved by the Animal Welfare Committee of the Institute of Zoology, Chinese Academy of Sciences. The experimental data are expressed as mean±standard deviation and analyzed with GraghPad Prism6 statistical software, *P<0.05, **P<0.01, ***P<0.001.

Human embryonic kidney cell 293T line: ATCC product, article No.: CRL-3216.

293T cell medium: based on the parts by volume, 89 parts of DMEM high-sugar medium (Hyclone), 10 parts of fetal bovine serum (Gibco, 10100-147), and 1 part of penicillin/streptomycin (Invitrogen, 15070-063).

Mesenchymal precursor cell medium (i.e. MPC medium): based on parts by volume, 88 parts of α-MEM+GlutaMAX (Invitrogen, 12571071), 10 parts of fetal bovine serum (Gibco, 10100-147), 1 part of non-essential amino acids (Invitrogen, 11140-050), 1 part of penicillin/streptomycin (Invitrogen, 15070-063); a recombinant human fibroblast growth factor (JPC) is added, with a concentration of 1 ng/ml in the system.

The WS mesenchymal precursor cells in the following embodiments are the mesenchymal stem cells with function lost WRN in a Chinese patent (Multipotent Stem Cells Carrying Human progeria Gene Mutations and Preparation Method Thereof, No. ZL201510137846.4). The biological material can be obtained from the applicant, and the biological material is only used for repeating the relevant experiments of the present invention and cannot be used for other purposes.

Mesenchymal precursor cells were prepared from a cell line of human embryonic stem cells H9 (WiCell product, article No.: WA09(H9)-DL-7), and the specific steps are as follows:
(1) human embryonic stem cells H9 were subject to the differentiation of an embryoid body to obtain an embryoid body;
(2) the embryoid body obtained in step (1) was cultured until fibrous cells occurred;
(3) on completion of step (2), cells were collected, and then passaged and cultured;
(4) on completion of step (3), cells of which CD73, CD90 and CD105 were all positive, and were screened as the mesenchymal precursor cells.

The mesenchymal precursor cells were cultured according to the following method: the mesenchymal precursor cells were cultured with the above-mentioned mesenchymal precursor cell medium, and the cells were passaged once every 4-5 days. Each passage of cells was inoculated into one well of a 6-well plate at a density of 1×10⁵ cells.

In the embodiment below, the SA-β-gal staining for detecting cell senescence was performed according to the following steps:

SA-β-gal (senescence-associated beta-galactosidase) is a hydrolase in lysosomes and has enhanced activity in senescent cells; when X-Gal is added as a substrate, the SA-β-gal can enzymatically hydrolyze it and make cells appear blue. The SA-β-gal has thus become a “gold standard” for rapid detection of cell senescence. The specific steps are as follows: 1) mesenchymal precursor cells were inoculated into one well of a gelatin (sigma)-coated 6-well plate at a density of 1×10⁵ cells/well, and the cells were stained on D2; 2) on completion of step 1), the cells were fixed with a fixative solution (based on a volume percent, containing 2% (v/v) formaldehyde+0.2% (v/v) glutaraldehyde+97.8% (v/v) PBS) for 4 min (must not exceed 5 min), and then washed with PBS for twice; 3) on completion of step 2), 2 mL of staining solution (40 mM of citric acid/sodium phosphate buffer, 5 mM of K₄[Fe(CN)₆], 5 mM of K₃[Fe(CN)₆], 150 mM of NaCl, 2 mM of MgCl₂ and 1 mg/mL X-gal) was added to each well, and the cells were incubated overnight in the dark in a 37° C. bacterial incubator; and 4) on completion of step 3), the cells were washed with PBS for twice, and then observed and photographed under an inverted microscope.

### Example 1. The Knockdown KAT7 Genes can Delay an Accelerated Senescent Phenotype of Mesenchymal Precursor Cells Carrying Human Progeria Gene Mutation

The present invention finds that the knock=down KAT7 (lysine acetyltransferase 7) genes can delay an accelerated senescent phenotype of mesenchymal precursor cells carrying human progeria gene mutation, and the experimental steps were as follows:

1. Preparing Recombinant Lentivirus for Knocking Down Human KAT7 Genes

1.1 An sgRNA sequence of knockdown KAT7 gene was obtained from a GeCKOv2.0 (1000000048) plasmid library, as shown below:

sgKAT7-huamn-F1:
5′-CACCGTTCAATCTCTGTGTTTGAAG-3′
(sequence 1 in a sequence list);
sgKAT7-human-R1:
5′-AAACCTTCAAACACAGAGATTGAAC-3′
(sequence 2 in the sequence list);
sgKAT7-huamn-F2:
5′-CACCGACGATCTGCTCGAGTCACCC-3′
(sequence 3 in a sequence list);
sgKAT7-human-R2:
5′-AAACGGGTGACTCGAGCAGATCGTC-3′
(sequence 4 in the sequence list);

An oligonucleotide sequence of the above gRNA targeting the KAT7 genes was synthesized in the company (Thermo Fisher). sgKAT7-huamn-F1 and sgKAT7-human-R1 were annealed to obtain sgKAT7-human-1, a T4 ligase (NEB) was used for ligating the sgKAT7-human-1 to a vector backbone obtained by digesting lenti-CRISPRv2 (Addgene product, #52961) with FastDigest_Esp3I (NEB), and the obtained recombinant vector with a correct sequence was recorded as a recombinant vector sgKAT7-human-1. The lenti-CRISPRv2 vector backbone contains a Cas9 endonuclease encoding gene and can express the Cas9 endonuclease; and it also contains an insertion site of exogenous DNA fragments for guiding Cas9 to a specific site in a genome, as well as the coding DNA of the gRNA backbone. The recombinant vector sgKAT7-human-1 can encode sgRNA that directs KAT7 genes.

According to the above method. sgKAT7-huamn-F2 and sgKAT7-human-R2 were annealed to obtain sgKAT7-human-2, a T4 ligase (NEB) was used for ligating the sgKAT7-human-2 to a vector backbone of lenti-CRISPRv2 (Addgene product, #52961) digested with FastDigest_Esp3I (NEB), and the obtained recombinant vector with a correct sequence was recorded as a recombinant vector sgKAT7-human-2. The recombinant vector sgKAT7-human-2 can encode sgRNA that guides KAT7 genes.

According to the above method, sgNTC-F1 and sgNTC-R1 were annealed to obtain sgNTC, a T4 ligase (NEB) was used for ligating the sgNTC to a vector backbone obtained by digesting lenti-CRISPRv2 (Addgene product, #52961) with FastDigest_Esp3I (NEB), and the obtained recombinant vector with a correct sequence was a control vector.

sgNTC-F1:
5′-CACCGACGGAGGCTAAGCGTCGCAA-3′ (sequence 5 in
the sequence list);
sgNTC-R1:
5′-AAACTTGCGACGCTTAGCCTCCGTC-3′ (sequence 6 in
the sequence list).

1.2. The lentiviral plasmid sgKAT7-human (i.e., the recombinant vector sgKAT7-human-1 or the recombinant vector sgKAT7-human-2), a lentiviral packaging vector psPAX2 (Addgene product, #12260) and pMD2G (Addgene product, #12259) were co-transfected with 293T cells to obtain sgKAT7-human virus; the steps were as follows:

1.2.1 By using a Lipo3000 transfection kit (ThermoFisher), the lentiviral plasmid sgKAT7-human, a lentiviral packaging vector psPAX2 and pMD2G were co-transfected with 293T cells (the ratio was: for one 10 cm dish 293T cell: 9 μg of the lentiviral plasmid sgKAT7-human, 6 μg of the psPAX2, and 3 μg of the pMD2G), and then cultured for 8 h.

1.2.2 On completion of 51.2.1, a fresh 293T cell medium was replaced to culture for another 48-54 h.

1.2.3 On completion of S1.2.2, the supernatant was collected and filtered with a 0.22 μm filter membrane, and the filtrate was collected.

1.2.4 The filtrate obtained in S1.2.3 was centrifuged at 4° C. and at 19,400 rpm for 2.15 h, the supernatant was discarded, and the deposit was resuspended in an MPC medium to obtain a virus solution containing the sgKAT7-human recombinant lentivirus (called as sgKAT7-human virus solution for short); the virus solution prepared from the recombinant vector sgKAT7-human-1 was recorded as sg-KAT7 #1 virus solution, and the virus solution prepared from the recombinant vector sgKAT7-human-2 was recorded as sg-KAT7 #2 virus solution.

According to the method in S1.2, the lentiviral plasmid sgKAT7-human was replaced by the control vector, and other steps remained unchanged to obtain a control virus solution which was recorded as sgNTC virus solution.

2. Infecting Mesenchymal Precursor Cells with the sgKAT7-Human Lentivirus

The passage-4 WS mesenchymal precursor cells were used as test cells which were infected with the sgNTC and the two types of sgKAT7-human viruses, respectively. The specific method was as follows: 2 μL of the sgKAT7-human lentivirus (or the sgNTC virus) and 2 μL of Polybrene were added to a culture well (1 well of a 6-well plate) inoculated with passage-4 WS mesenchymal precursor cells. The medium was changed on the next day, and then the cells were cultured normally and passaged.

After infected with the sgNTC or sgKAT7-human lentiviruses, the cells obtained were successively passaged to the passages 3-4. The cells were collected, the KAT7 protein knockdown efficiency was detected, and SA-β-gal staining of cell senescence markers was performed to detect the mRNA levels of IL6, a cell proliferation molecular marker (Ki67) and senescence-associated genes (p16^{INK4A}, p21^{CIP1}, LMNB1 and LAP2).

A reagent for detecting the Ki67 is a product of ZSGB-BIO, with an article No. of ZM-0166.

Primers for detecting the mRNA levels of p16^{INK4A}, p21^{CIP1}, LMNB1 and LAP2 are as follows:

p16^{INK4A}-F1:
5′-ATGGAGCCTTCGGCTGACT-3′ (sequence 7 in the
sequence list);
p16^{INK4A}-R1:
5′-GTAACTATTCGGTGCGTTGGG-3′ (sequence 8 in the
sequence list);
p21^{CIP1}-F1:
5′-CGATGGAACTTCGACTTTGTCA-3′ (sequence 9 in the
sequence list);
p21^{CIP1}-R1:
5′-GCACAAGGGTACAAGACAGTG-3′ (sequence 10 in the
sequence list);
LMNB1-F1:
5′-GAAAAAGACAACTCTCGTCGCA-3′ (sequence 11 in the
sequence list);
LMNB1-R1:
5′-GTAAGCACTGATTTCCATGTCCA-3′ (sequence 12 in the
sequence list);
LAP2-F1:
5′-CCCCTCGGTCCTGACAAAAG-3′ (sequence 13 in the
sequence list);
LAP2-R1:
5′-CGCTCTTCGTCACTGGAGAA-3′ (sequence 14 in the
sequence list).

A reagent for detecting IL6 secretion is a product of BioLegend, with an article No. of 504601.

An antibody for detection an expression of a KAT7 protein is a product of Abcam, with an article No. of ab70183.

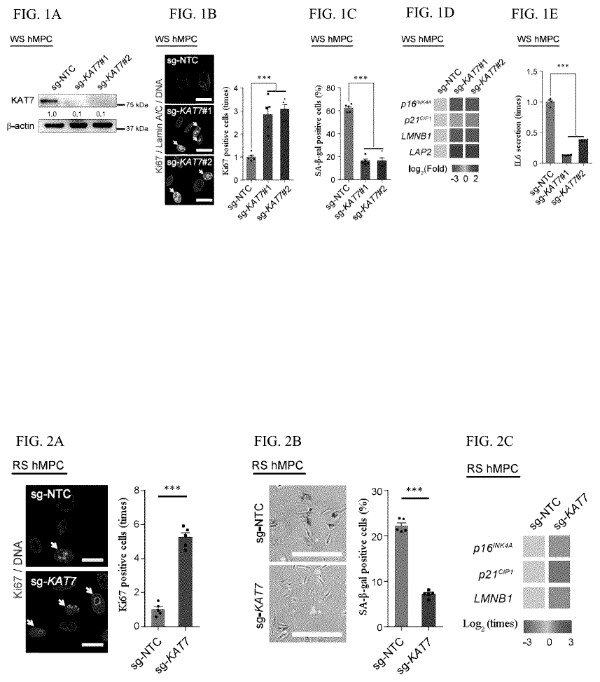

The results of biological research methods for senescence such as detection of senescence-associated SASP (FIGS. 1A-1E) show that compared with the sgNTC lentivirus-infected cells in the control group, the expressions of the KAT7 protein in sgKAT7-human lentivirus-infected cells decrease (FIG. 1A), the numbers of Ki67-positive cells increase significantly, and the numbers of the Ki67-positive cells in the cells infected with the two sgKAT7-human lentiviruses are 2.9 and 3.1 times of that in the sgNTC lentivirus-infected cells, respectively (FIG. 1B); the numbers of SA-β-gal stained positive cells in the sgKAT7-human lentivirus-infected cells decrease significantly, and the numbers of SA-β-gal positive cells in the cells infected with the two sgKAT7-human lentiviruses are and 0.3 times of that in the sgNTC lentivirus-infected cells, respectively (FIG. 1C); the expressions of P16^{INK4A} and p21^{CIP1} in the sgKAT7-human lentivirus-infected cells significantly decrease, and the expressions of LMNB1 and LAP2 significantly increase (FIG. 1D); the IL6 secretion in the sgKAT7-human lentivirus-infected cells significantly decreases, and the IL6 secretion in the cells infected with the two sgKAT7-human lentiviruses is 0.1 and times of that in the sgNTC lentivirus-infected cells, respectively (FIG. 1E).

It is indicated that the sgKAT7-human lentivirus can significantly delay a pathological senescent process of WS mesenchymal precursor cells.

### Example 2: Knockdown KAT7 Genes can Delay the Replicative Senescence of Mesenchymal Precursor Cells

The passage-7 mesenchymal precursor cells were used as test cells which were infected with the sgNTC virus and the sgKAT7-human virus (sg-KAT7 #1 lentivirus) obtained in example 1, respectively, and the preparation method and the infection method for the lentiviruses were the same as those in example 1. The cells infected with the viruses were collected after 3-4 passages to perform SA-β-gal staining of cell senescence markers and detect the mRNA levels of a cell proliferation molecular marker (Ki67) and senescence-associated genes (p16^{INK4A}, p21^{CIP1}, LMNB1), and the detection method was the same as that in example 1. The method for detecting the expression level of the KAT7 protein was the same as that in example 1.

The results show that the expressions of the KAT7 protein in the sgKAT7-human lentivirus-infected cells decrease, and the growth of the sgNTC-infected mesenchymal precursor cells is completely blocked after 3-4 passages, while the sgKAT7-human virus-infected mesenchymal precursor cells can grow rapidly (the number of Ki67-positive cells in the sgKAT7-human virus-infected cells is 5.2 times of that in the sgNTC lentivirus-infected cells, (FIG. 2A); the results of the SA-β-gal staining for cell senescence markers, the detection of cell proliferation ability and the RT-qPCR detection of senescence molecular markers such as p16^{INK4A} and p21^{CIP1} show that compared with the sgNTC lentivirus, the sgKAT7-human lentivirus can significantly delay the replicative senescence of mesenchymal precursor cells (FIGS. 2A-2C): the number of SA-β-gal positive cells in the sgKAT7-human lentivirus-infected cells is 0.4 times of that in the sgNTC lentivirus-infected cells (FIG. 2B); compared with the sgNTC lentivirus-infected cells, the expressions of p16^{INK4A} and p21^{CIP1} significantly decrease and the expression of LMNB1 significantly increases in the sgKAT7-human lentivirus-infected cells (FIG. 2C).

### Example 3: Therapeutic Effect of Knockdown KAT7 Genes on Delaying Senescence in Aged Mice

1. Preparing Lentivirus for Knocking Down Mouse KAT7 Genes

1.1 Preparing a Recombinant Vector

sgKAT7-mouse-F:
5′-CACCGAGCCGCCGGCAATGCCGCGA-3′
(sequence 15 in the sequence list);
sgKAT7-mouse-R:
5′-AAACTCGCGGCATTGCCGGCGGCTC-3′ (sequence
16 in the sequence list).

An oligonucleotide sequence of the above gRNA targeting the KAT7 genes was synthesized in the company (Thermo Fisher). sgKAT7-mouse-F and sgKAT7-mouse-R were annealed to obtain sgKAT7-mouse, and a T4 ligase (NEB) was used for ligating sgKAT7-mouse to a vector backbone obtained by digesting lenti-CRISPRv2 (Addgene product, #52961) with FastDigest_Esp3I (NEB), accordingly constructing a sgKAT7-mouse lentiviral plasmid with a correct sequence and capable of specifically knocking down mouse KAT7 genes. The sgKAT7-mouse lentiviral plasmid could be used for knocking down mouse KAT7 genes.

1.2. The lentiviral plasmid sgKAT7-mouse and the lentiviral packaging vectors of psPAX2 and pMD2G were used for co-transfecting 293T cells to obtain a sgKAT7-mouse virus according to step 1.2 in example 1, and then a control virus (sgNTC) was obtained. Both viruses were resuspended with 1×PBS, and the viral loads in the two viral solutions were the same.

2. Establishing Normal Mice Models of Natural Aging

Male 20-month-old SPF mice (C57BL6) were provided by Suzhou AIERMAITE Technology Co., Ltd. Mice feeding conditions include 23° C., 12 h light/12 h darkness, and free access to food and water. Experimental equipment and materials include an isoflurane gas anesthetic apparatus and insulin syringes.

2.1 Grouping and Treatment

sgKAT7 group (17 model mice): on D1 and D60 of the experiment, each mouse was injected with 100 μl of the sgKAT7-mouse lentiviral solution via a caudal vein, respectively.

sgNTC group (17 model mice): on D1 and D60 of the experiment, each mouse was injected with 100 μl of the sgNTC-mouse lentiviral solution via a caudal vein, respectively.

Young group (6 SPF mice (C57BL6)): the 2-month-old mice were fed normally without treatment.

Aged group (6 SPF mice (C57BL6)): the 28-month-old mice were fed normally without treatment.

3. Effect Evaluation

The therapeutic effects were evaluated after 6 months of the experiment. The specific method is as follows:

3.1. Detection of sgKAT7-Mouse Knockdown Efficiency Using Western Blot

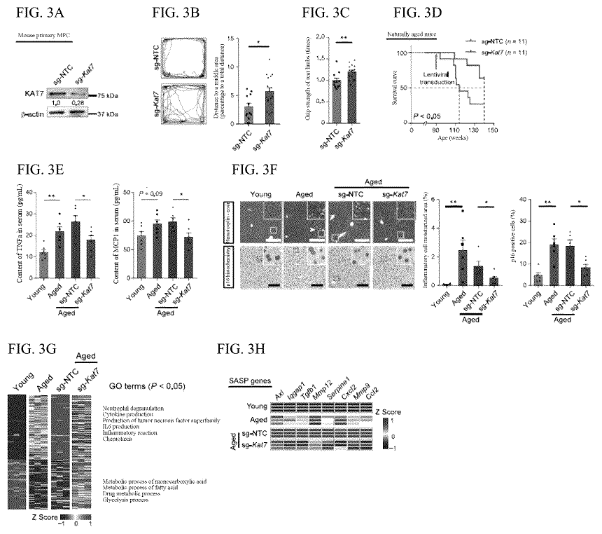

Before an in vivo experiment on the mice, mesenchymal precursor cells (MPCs) isolated from bone marrow of the mice (C57BL6) were infected with the sgKAT7-mouse lentivirus and the control virus (sgNTC), respectively, and proteins were extracted after 7 days to detect KAT7 expressions using western blotting and identify the sgKAT7-mouse knockdown efficiency (FIG. 3A). The results show that the sgKAT7-mouse lentivirus can reduce a KAT7 content in cells.

Antibody: Anti-human/mouse KAT7 antibody (ab70183), Abcam.

Anti-human actin antibody (sc-69879), Santa Cruz Biotechnology.

3.2 Behavioral Detection of Senescence-Associated Degenerative Changes

Six months after the aged mice were injected with the virus, the health conditions of the mice were examined using an open field test and a grip strength test.

Open field test: first, an open black wooden box was prepared (the internal field was 81 cm×81 cm in size and 28 cm in height. A bottom of the box was vertically and horizontally painted with 3 white lines (3 mm wide), respectively to form 16 squares of equal size (each square was 20 cm×20 cm). After that, each mouse was tested once; the mouse was placed in one of the 4 corner squares facing a wall, and it explored the environment freely for 5 min. The time and distance for the mouse in the 4 squares in the middle were analyzed using software.

Grip strength test: the grip strength of each mouse's forelimbs was measured; the mouse's tail was grabbed and then pulled backward when its forelimbs grabbed a grip tester, a reading of the grip tester was read, and each mouse was tested for 10 times to take an average value.

The results (FIGS. 3B-3D) find that the old mice injected with the sgKAT7-mouse virus performed better, and it is found by continued feeding that the life of the mice injected with the sgKAT7-mouse virus is prolonged. At the age of 143 weeks, the mice injected with the sgKAT7-mouse virus and the control virus (sgNTC) have a median survival time of 143 weeks and 114 weeks, respectively.

3.3 Detection of Inflammatory Factors in Mouse Blood

Eight months after the aged mice were injected with the virus, 6 mice in each group were killed, and serum was extracted to detect the contents of inflammatory factors such as a tumor necrosis factor TNFa and a monocyte chemotactic protein MCP1 in the serum. The results indicate that compared with the mice injected with the control virus, the contents of TNFa and MCP1 in the serum of the aged mice injected with the gKAT7-mouse virus are significantly reduced (FIG. 3E)). The young group and the aged group are used as controls.

The TNFa and MCP1 were detected using conventional methods, with the service provided by Beijing Zhongyuan Ltd.

3.4. Staining of Mouse Liver Tissues

An important indicator for an improvement in the state of aged mice is a reduction in inflammatory infiltrating cells in a liver tissue and a reduction in a proportion of senescent cells. Eight months after the aged mice were injected with the virus, the liver tissues of the mice were stained, and the young group and the aged group were used as controls.
(1) A mouse was perfused with normal saline until the liver became relatively white, and a part of the liver tissue was taken.
(2) The liver tissue was fixed with 4% paraformaldehyde (PFA) for 1 week.
(3) The liver tissue was treated with a 30% sucrose solution for 2 weeks and then dehydrated.
(4) The liver tissue was paraffin-embedded and sectioned. The slices were 5 mm thick.
(5) The slices were dewaxed and rehydrated.
(6) The slices were stained with iron hematoxylin (ZSGB-BIO, ZLI-9610) for 8 min, and then washed with running water for 2 min.
(7) The slices were stained with 0.5% eosin (ZSGB-BIO, ZLI-9613) for 2 min.
(8) The slices were dehydrated, mounted and observed under a microscope.

When no virus was injected, compared with those in the young group, the inflammatory cells in the mouse liver tissue increased in the aged group. Compared with those in the group in which the mice were injected with the sgNTC virus, the inflammatory cells in the mouse liver tissue were reduced. In addition, p16 staining indicates a reduction in senescent cells (FIG. 3F).

3.5 RNA-seq Analysis on Liver Tissues of Aged Mice

After 8 months, the mice were harvested, and a part of each liver tissue was extracted with TRIZOL (gibco, 15596018) and sent to the company (Nuohe) to build a library for transcriptome RNA-seq sequencing. Bioinformatic analysis indicates that up-regulated genes in the liver of aged mice are mainly concentrated in an inflammatory response, and down-regulated genes were mainly concentrated in a metabolic response. It is found that KAT7 knockdown can reverse about one-third of the genes that are altered in the liver tissue of aged mice, and most of these reversed genes are concentrated in inflammation and metabolism. Besides, it is also found that the expression of some SASP-associated genes will be down-regulated after KAT7 knockdown (FIGS. 3G-3H).

### Example 4: Knockdown KAT7 Genes can Delay the Senescence of Human Primary Hepatocytes

Human primary hepatocytes were used as test cells (a product of Lonza, with an article No. of HUCPI) to be infected with the sgNTC virus and the sgKAT7-human virus obtained in example 1, respectively, and the preparation method and the infection method of the lentiviruses were the same as those in example 1. The virus-infected cells were continuously cultured for 8 days, and a medium is produced by Lonza, with an article No. of CC-3198.

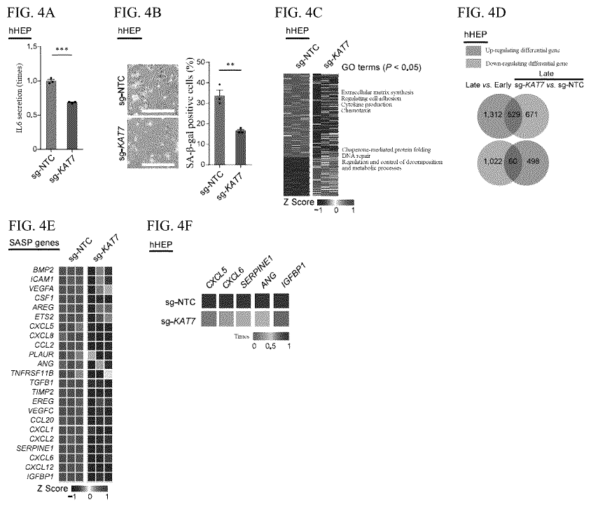

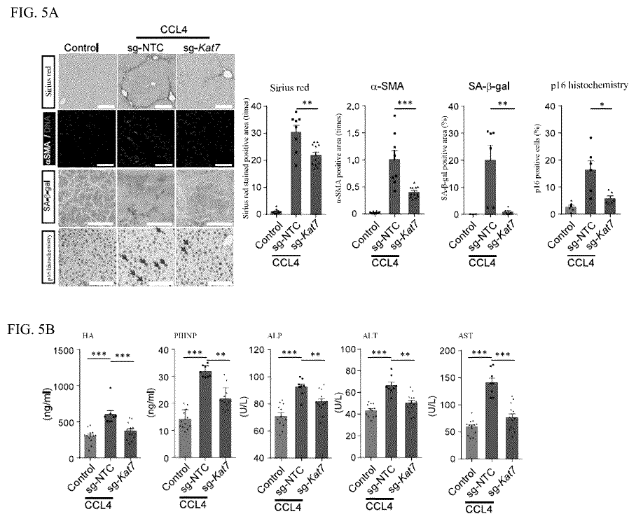

After the culture, the detection results of the SA-β-gal staining of cell senescence markers and IL6 in cell culture supernatant show that compared with the sgNTC lentivirus, the sgKAT7-human lentivirus can significantly delay the senescence of hepatocytes (FIGS. 4A-4B). Besides, RNA-seq analysis shows a consistent result with that of the liver tissues of aged mice after KAT7 knockdown, the expressions of a quantity of inflammatory genes and SASP-associated genes are down-regulated, and the expressions of metabolism-related genes are up-regulated. These results indicate that knockdown KAT7 can delay the senescence of primary hepatocytes (FIGS. 4C-4F).

### Example 5: Therapeutic Effect of Knockdown KAT7 Genes on Delaying CCL4-Induced Hepatic Fibrosis

1. Establishing Mice Models of CCL4-Induced Hepatic Fibrosis

Male 10-week-old SPF mice (C57BL6), weighing 20±2 g, were provided by Suzhou AIERMAITE Technology Co., Ltd. Mice feeding conditions include 23° C., 12 h light/12 h darkness, and free access to food and water. Experimental equipment and materials include an isoflurane gas anesthetic apparatus, surgical scissors, scalpels, suture, penicillin, an asanascope and insulin syringes.

Days were continuously counted in step 1.1, step 1.2 and step 1.3.

1.1. Establishing Mice Models of CCL4-Induced Hepatic Fibrosis

Surgeries for CCL4-induced hepatic fibrosis were performed on D1 of the experiment. A surgical method was as follows: firstly, a mouse was anesthetized, and an abdomen of the mouse was depilated with an epilator; 10% CCL4 oil solution (CCL4: olive oil=1:9 based on a volume percentage) was injected intraperitoneally according to 1 ml/kg body weight, twice a week. The mice were continuously injected for 8 weeks. The mice after the surgery of CCL4-induced hepatic fibrosis were named as model mice. The animals in the control group were intraperitoneally injected with the same dose of an olive oil solvent.

The mice not treated by surgery were named as normal mice.

1.2 Grouping and Treatment

sgKAT7 group (12 model mice): at week 3, 200 μl of the sgKAT7-mouse lentivirus in example 3 was injected via a hepatic portal vein, and an outer skin was tightly sutured; penicillin was applied to the surgical site to prevent infection.

sgNTC group (8 model mice): at week 3, 200 μl of the sgNTC lentivirus in example 3 was injected via a hepatic portal vein, and an outer skin was tightly sutured; penicillin was applied to the surgical site to prevent infection.

Normal group (12 normal mice, control): no treatment was given.

1.3 Effect Evaluation

The therapeutic effect on CCL4-induced hepatic fibrosis was evaluated at week 8 of the experiment. The specific method is as follows:

1.3.1 Sirius Red Staining of Mouse Liver

One of the manifestations of hepatic fibrosis in mice is a diffuse excessive deposition and abnormal distribution of collagen and other extracellular matrix in the liver. At week 8 after CLL4-induced hepatic fibrosis modeling, the mice were killed, and a part of a hepatic lobule was taken for section staining.
(1) A mouse was perfused with normal saline until the liver became relatively white, and then a part of the liver tissue was taken.
(2) The liver tissue was fixed with 4% paraformaldehyde (PFA) for 1 week.
(3) The liver tissue was treated with a 30% sucrose solution for 2 weeks and then dehydrated.
(4) The liver tissue was paraffin-embedded and sectioned. The slices were 5 mm thick.
(5) The slices were dewaxed and rehydrated.
(6) The slices were stained with iron hematoxylin (ZSGB-BIO, ZLI-9610) for 8 min, and then washed with running water for 2 min.
(7) The slices were stained with a sirius red staining solution (ZSGB-BIO, DC0041-2) for 1 h.
(8) The slices were dehydrated, mounted and observed under a microscope.

Compared with that in the normal group, the sirius red staining of the mice in the sgNTC group was significantly deepened. Compared with that in the sgNTC group, the sirius red staining was attenuated in the sgKAT7-mouse group. Furthermore, compared with those in the sgNTC group, SA-b-gal and p16-positive cells of senescence-associated staining are found to be reduced (FIG.

1.3.2 Liver Function Test by Mouse Blood

After the mouse was killed, blood was taken from an eyeball to detect contents of some factors characterizing liver functions, such as hyaluronic acid (HA), procollagen type III N-terminal peptide (PIIINP), alkaline phosphatase (ALP), (alanine aminotransferase) ALT and aspartate Transaminase (AST). The results show that compared with those in the normal group, the factors characterizing the function of hepatocytes were down-regulated, and the expressions were up-regulated in the sgKAT7-mouse virus-infected group (FIG. 5B).

The contents of HA, PIIINP, ALP, ALT and AST were all detected using conventional methods, with the service provided by Beijing Zhongyuan Ltd.

It is indicated that knockdown KAT7 genes have a therapeutic effect on delaying CCL4-induced hepatic fibrosis.

### Example 6: Effect of WM-3835 as a KAT7 Inhibitor on Delaying the Senescence of WS hMPCs and Hepatocytes

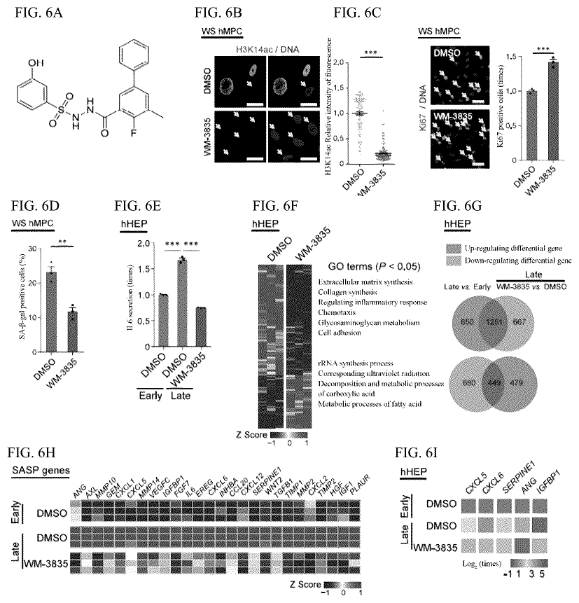

Passage-4 WS mesenchymal precursor cells (WS hMPCs) in example 1 were used as test cells, and the cells were treated with a DMSO solution of 50 nM of WM-3835. The WM-3835 (with a structural formula as shown in a of FIGS. 6A-6I) was synthesized by entrusting WuXi AppTec, and a DMSO solvent was used as a control.

After the treatment, the cells were continuously passaged to passages 2-3. The cells were collected to detect a KAT7 protein knockdown efficiency (FIG. 6B), and SA-β-gal staining of cell senescence markers and detection of a cell proliferation molecular marker (Ki67) were performed. It is found that the inhibitor WM-3835 can delay the senescence of WS hMPCs (FIGS. 6C-6D).

Human primary hepatocytes (hHEPs) were used as test cells (a product of Lonza, with an article No. of HUCPI), and the cells were treated with a DMSO solution of 100 nM of WM-3835, and DMSO was used as a control.

The cells were cultured for 8 d after the treatment, the senescence molecular markers such as P15 and IL6 were detected using RT-qPCR and the detection results show that compared with that in the DMSO group, WM-3835 can significantly delay the senescence of hepatocytes. Besides, RNA-seq analysis shows a consistent result with that of the KAT7 knockdown using the sgKAT7-human lentivirus after the WM-3835 treatment, the expressions of a quantity of inflammatory genes and SASP-associated genes are down-regulated, and the expressions of metabolism-related genes are up-regulated. These results indicate that WM-3835 can delay the senescence of primary hepatocytes (FIGS. 6E-6I, wherein Early represents a result of an early passage (at D4 of treatment), and Late represents a result of a late passage (at D9 of treatment)).

### INDUSTRIAL APPLICATION

The experiments of the present invention confirm that knockdown KAT7 genes have a definite effect on delaying senescence and treating hepatic fibrosis; WM-3835 as a KAT7 inhibitor has a definite effect on delaying the senescence of mesenchymal precursor cells and hepatocytes. The present invention provides a new idea for developing genes for delaying senescence and treating hepatic fibrosis, and expands the options of clinical gene therapy and drug therapy.

1-18. (canceled)

19. Any of the following methods:
(i) a method in preparing a product for treating and/or preventing senescence;
(ii) a method in preparing a product for inhibiting hepatic fibrosis.

20. The method of claim 19, wherein the method in preparing a product for treating and/or preventing senescence comprises the use of any of the following substances;
(iii) a substance that reduces the content or activity of KAT7;
(iv) a substance that inhibits KAT7 gene expressions or knocks down KAT7 genes.

21. The method of claim 20, wherein the substance is a CRISPR/Cas9 gene knockout system targeting KAT7 genes.

22. The method of claim 21, wherein the substance is a recombinant vector containing the CRISPR/Cas9 gene knockout system.

23. The method of claim 20, wherein the substance is a KAT7 inhibitor.

24. The method of claim 23, wherein the KAT7 inhibitor is WM-3835.

25. The method of claim 19, wherein the senescence is that of an animal cell, a tissue, an organ or an individual.

26. The method of claim 19, wherein the method in preparing a product for inhibiting hepatic fibrosis comprises the use of a substance that reduces the content or activity of KAT7 or a substance that inhibits KAT7 gene expressions or knocks out KAT7 genes.

27. Any of the following products:
(v) a product for treating and/or preventing senescence;
(vi) a product for inhibiting hepatic fibrosis.

28. The product according to claim 27, wherein an active ingredient of the product for treating and/or preventing senescence is a substance that reduces the content or activity of KAT7 or a substance that inhibits KAT7 gene expressions or knocks out KAT7 genes.

29. The product according to claim 27, wherein an active ingredient of the product for inhibiting hepatic fibrosis is a substance that reduces the content or activity of KAT7 or a substance that inhibits KAT7 gene expressions or knocks out KAT7 genes.

30. A method for delaying and/or treating and/or preventing senescence, comprising the following steps: administrating a substance that reduces the content or activity of KAT7 or a substance that inhibits KAT7 gene expressions or knocks out KAT7 genes on an animal cell, a tissue, an organ or an individual, in order to delay and/or treat and/or prevent the senescence of the animal cell, the tissue, the organ or the individual.

31. The method of claim 30, wherein the animal is a mammal.

32. The method of claim 30, wherein the mammal is a human or a mouse.
